(19) United States
(12) Patent Application Publication
(10) Pub. No.: US 2005/0079484 A1
(43) Pub. Date: Apr. 14, 2005

(54) Method of detecting biological materials in liquid

(76) Inventors: William Richard Heineman, Cincinnati, OH (US); Hallen Brian Halsall, Cleves, OH (US); Carl James Seliskar, Blue Ash, OH (US)

Correspondence Address: FROST BROWN TODD, LLC, 2200 PNC CENTER, 201 E. FIFTH STREET, CINCINNATI, OH, 45202, US

(21) Appl. No.: 10/683,846

(22) Filed: Oct. 10, 2003

(51) Int. Cl. C12Q 1/70; G01N 33/554; G01N 33/569

(52) U.S. Cl. 435/5; 436/125; 435/7.32

(57) ABSTRACT

The present invention provides a method of detecting targeted agents in liquid, and in particular, the detection of targeted biological agents in finished water and other liquids. The methods disclosed herein can be used for the detection of biological agents that can be used as bioterrorism agents in a bioterrorism attack.

### FIELD OF THE INVENTION

The present invention relates to the detection of a targeted agent in liquid, and in particular, the detection of biological agents in water that is within a water supply system, and most particularly, the present invention relates to a method of detecting biological agents in finished water.

### BACKGROUND OF THE INVENTION

Water, an essential resource, should be monitored to ensure that it is safe for human contact and consumption. As such, our water system, including lakes, streams, surface water, groundwater, and any other water that humans are exposed to, or becomes part of the water supply system, should be monitored and protected. As used herein, a water supply system refers to waterworks, pumping stations, treatment stations, storage facilities, and the like. Storage facilities, for example, provide extra water reserves for use when demand is high or, when necessary, to help maintain water pressure. Treatment stations, for example, are facilities where water may be filtered to remove suspended impurities, aerated to remove dissolved gases, or disinfected with an agent that kills harmful bacteria, or other microorganisms. Further, not all water supply systems are used to deliver drinking water. Systems used for purposes such as irrigation and fire fighting operate in much the same way as systems for drinking water, but the water need not meet such high standards of purity. It is, however, just as important that those water systems be monitored and protected from exposure to potentially harmful biological agents.

As used herein, monitoring refers to a process or methodology in practice that can recognize and give warning to the presence of a potentially harmful and/or lethal agent in a liquid, such as, for example, a water supply. In providing an effective monitoring system, various characteristics should be taken into consideration. For example, qualities such as speed of analysis, low limits of detection, and accuracy should be considered. A fast analysis can be advantageous because it facilitates rapid detection of any possible targeted agent so that both the dispersal of the agent in a distribution and/or supply system and exposure to the consumer is minimized. A low limit of detection for biological agents that are possibly toxic or infectious bioterrorism agents and accuracy are helpful in preventing false negatives or positives, as well as maintaining confidence in the monitoring methodology.

Such monitoring and protection is indispensable in ensuring that the water people are coming into contact with, and/or consuming is safe and free from contaminants that could cause health problems, such as, for example, naturally present biological agents, such as bacteria or other microorganisms. Such contaminants can cause sickness or other long-term damage for those who ingest, or otherwise contact the contaminated water. Further, recent events have brought the vulnerability of the Nation's resources to the forefront of our consciousness. The water we consume is particularly troublesome because, although it may not be practical to monitor all the numerous possible entry points into a distribution grid between a treatment plant and a consumer, these are the most obvious places to introduce high concentrations of dangerous biological agents for the best chance of a successful attack.

One challenge, in particular, for a biological agent detection system is to be able to discern a specific signal from a targeted biological agent while rejecting, or minimizing, signals originating from the nonpathogenic (nontoxic) biological background. Biological detection systems are currently only in research and early development stages. Although there are some commercially available devices, they have limited utility in that they only respond to a small number of agents and are generally high cost items. This is in stark contrast to chemical detection equipment where there are multiple technologies available for purchase that can detect chemical agents and/or toxic industrial materials.

One reason for the unavailability of biological detection equipment is that detection of biological agents requires high sensitivity (because of the very low effective dose needed to cause infection and spread the disease), as well as a high degree of selectivity (because of the large and diverse biological background in the environment). Further, biological agents, in comparison to chemical agents, are very complex systems of molecules, which can make identification difficult. For example, Ionization/Ion Mobility Spectrometry (IMS), an excellent, though expensive system for collection, detection, and identification of chemical agents, cannot detect or discriminate biological agents in its present form. In fact, the need for high-efficiency collection and concentration of the sample, high sensitivities, and high selectivities make all chemical detectors in their current form unusable for biological agent detection.

Further, the complexities of finished water makes any performing assays on the water difficult. In particular, the additives included to disinfect prepare the water for contact with consumers can interfere with, and in some cases, prevent the assay from properly working to identify the targeted biological agent.

Consequently, a significant need exists for a viable method of detecting biological agents, and in particular, detecting biological agents in water or other liquids. There is a further need for a method of selectively monitoring a water supply to determine the presence of biological agents, and in particular biological agents that can act as bioterrorism agents in finished water.

The present invention addresses these and other problems by providing a novel method of detecting biological agents in finished water.

### BRIEF SUMMARY OF THE INVENTION

The present invention provides a method of detecting biological agents in finished water including the steps of providing a first molecular recognition element manipulated to target a biological agent in finished water; flowing at least one sample suspected of having the biological agent over the first recognition element; capturing the biological agent present in the sample with the first recognition element; and emitting a signal capable of indicating the presence of the targeted biological agent in the sample.

The method can further include the step of associating at least one second recognition element that is manipulated to target the captured biological agent, wherein either the first molecular recognition element or the second molecular recognition element is capable of being manipulated to emit a signal indicating the presence of the targeted biological agent in the sample. Additionally, the method of the present invention can include a signal that is capable of electrochemical detection, as well as a signal that is capable of fluorescence detection.

The present invention further provides a method of detecting the presence of a biological agent in finished water, comprising the steps of (a) providing a finished water sample; and (b) adjusting the environmental conditions of the finished water sample of interest by combining the finished water sample with an antigen diluent or buffer comprising one or more compounds selected from the group consisting of a reducing agent, a buffering agent, a chelating agent, a blocking agent of non-specific binding, a chaotropic agent, an antibacterial agent, and a detergent; wherein the antigen diluent or buffer is present in a concentration sufficient to produce positives in the assay.

These and other objects and advantages of the present invention shall be made apparent from the accompanying drawings and the description thereof.

### DETAILED DESCRIPTION OF THE INVENTION

The present disclosure is directed to the detection of biological agents, and, in particular, biological agents that are harmful to humans or animals, and most particularly, to the detection of biological agents in finished water that are most likely to be used as bioterrorism agents in a bioterrorist attack.

Biological agents can be identified using multiple techniques, such as, for example immunoassays, which identify and measure a specific biological substance such as, for example, an antigen. Additionally, biological agents can be identified using a nucleic acid-based (NA-based) assay, which detects the specific agent by targeting a specific nucleic acid sequence. Both immunoassay and NA-based technologies operate, for example, by conducting molecular recognition events that target and capture a specific agent of interest. That capture is then translated into a signal that can be analyzed to determine the presence of the targeted agent. Although there are multiple ways to detect the presence of a targeted biological agent, two core technologies are most commonly involved with such detection: a molecular recognition event, and a transduction event that translates the recognition into a quantifiable signal.

Although the concepts of molecular recognition and signal emission in assays generally understood, conducting such assays is substantially different, and more complicated when the testing sample contains an intricate testing matrix with multiple constituents that can interfere with either recognition and/or signal emission of the targeted agent. An example of such a testing matrix is finished water.

Conducting an assay in finished water is a complicated endeavor. “Finished water” as used herein, refers to potable water that has been treated by a treatment plant, and is ready to be delivered to customers. When water is processed, for example, by a water works or other water provider, various additives to provide, for example, disinfection, nutrition, and/or maintenance of the water distribution system are often included.

Examples of disinfectants in water are, for example, chlorine, chloramines, chlorine dioxide, ozone, and ultra violet light, as well as others known in the art. Nutritional additives used are, for example, fluoride to prevent cavities and/or iodine to prevent goiter. Additionally, phosphates can also be added to control corrosion of the water distribution system.

Furthermore, finished water, depending on the source water's origin, contains various natural components. For example, by natural occurrence some waters are “hard,” and have high amounts of Ca²⁺ and/or Mg²⁺. Also, water, depending on its origin, contains varying pH and alkalinity (i.e., buffer capacity) levels. Therefore, the constituents present in finished water widely vary depending on the origin of the source water, as well as the water provider's treatment of the water.

The above-discussed natural qualities and/or additives present in finished water can interfere with the molecular recognition aspect of the assays disclosed herein. For example, the targeted agent can be altered so that the molecular recognition element can no longer recognize and/or capture the targeted agent. Additionally, the molecular recognition element can be altered so that it can no longer recognize and/or capture a targeted agent. By way of specific example, in a NA-based recognition system, various treatments, including, for example, chlorine, chloramine, chlorine dioxide, hypochlorite, and ozone can modify the nucleic acid sequence that identifies the targeted agent, and as such, can alter and/or disturb recognition on the targeted agent. As a result, although the targeted agent may not be infective, the loss in infectivity may not correlate with the RT-PCR titer. This could lead to false positives of infectivity, but true positives of presence.

Additionally, immunoassays rely on epitopes for recognition of the targeted agent. Epitopes, however, are vulnerable to modifications that can occur as a result of various constituents present in the water, such as, for example, disinfection agents (i.e., chlorine, chloramines, chlorine dioxide, hypochlorite, ozone), and residuals thereof. For example, oxidation due to the aquated chlorine or chloramines used for disinfection and maintained at residual levels in finished water can cause epitope alteration. As a further example, the amino acid side chains of tyrosine, tryptophan, cysteine, proline and histidine can also be modified by the addition of various disinfection agents. As a result, the alterations may render epitopes and/or nucleic acid sequences non-reactive towards the molecular recognition elements that have been developed for the unmodified version of the targeted agents.

As a further example, the presence of metal ions in a testing sample, such as calcium, magnesium, as well as other metals known in the art can react with the molecular recognition element and/or the targeted agent. The interference can be caused, for example, by the metal ions present coordinating with, for example, amine, sulfhydryl, histidyl, and/or carboxyl surface ligands. This interference, however, can be circumvented, for example, by adding a chelating agent that associates with the metal ions and renders them unable to interact with the molecular recognition element and/or the targeted agent. The term “chelating agent” as used herein refers to any organic or inorganic compound that will bind to a metal ion having a valence greater than one. A “chelator”, “chelating resin”, “binder”, “sequestration agent”, or “sequester of divalant cations” refers to a composition that binds divalent cations. The binding can be reversible or irreversible. Binding of the divalent cations generally renders them substantially unable to participate in chemical reactions with other moieties with which they come in contact. A “chelator”, “chelating resin”, “binder”, “sequestration agent”, or “sequester of divalant cations” refers to a composition that binds divalent cations. The binding can be reversible or irreversible. Binding of the divalent cations generally renders them substantially unable to participate in chemical reactions with other moieties with which they come in contact. Examples of chelating agents are, for example, ethylenediaminetetraacetic acid (EDTA), nitriloacetic acid (NTA), diethylenetriaminepentaacetic acid (DTPA), trans-1,2-diaminocyclohexanetetraacetic acid (DCTA), bis-(aminoethyl)glycoether-N,N,N′,N′-tetraacetic acid (ECTA), triethylene tetramine dihydrochloride (TRIEN), ethylene glycol-bis (beta.-aminoethyl ether)-N,N,N′,N′-tetracetic acid (EGTA), triethylenetetramine hexaacetic acid (TTG), deferoxamine, Dimercaprol, edetate calcium disodium, zinc citrate, penicilamine succimer, editronate as well as others known in the art. In one embodiment of the present invention the chelating agent has a concentration in the solution of between about 0.1 mM and about 50 mM. In another embodiment, the concentration of the chelating agent is between about 0.1 mM and about 10 mM. In another embodiment of the present invention, the chelator is provided in an amount such that the chelator comprises about 0.001M to about 0.05M, more preferably from about 0.005M to about 0.02M, and most preferably from about 0.008M to about 0.012M of the final chelator/finished water/(optional) buffer solution.

The chelator can be combined with finished water sample before, during, or after addition of the buffer mixture or acid to the finished water. Thus, for example, the chelator can be provided in the storage and/or preservation fluid provided with a finished water collection device. The chelator is then combined with the finished water during storage and transport. Alternatively, the chelator can be combined with the finished water just before application of the finished water sample to the assay device. In yet another embodiment, the chelator can be added to the assay device after application of the finished water or it can be stored in a reservoir within the assay device. In another embodiment, the chelator need not be combined, but only contacted with the finished water and/or the finished water/buffer mixture. Thus, for example where the immunoassay involves progression of the fluid through a porous matrix, the matrix material itself can be made of a material that chelates or otherwise sequesters or binds to divalent cations. Such matrix materials are well known to those of skill in the art. The most common sequestration agents are often used as ion exchange resins and include, but are not limited to chelex resins containing iminodiacetate ions, or resins containing free base polyamines, or amino-phosphonic acid. Alternatively, the finished water or finished water/buffer mixture can be pretreated by passage through a matrix that chelates or otherwise sequesters divalent cations. This pretreatment can be incorporated into the storage and transport container, provided as filtration step, or provided as a component of the method of extraction of the finished water sample from the collection device. In this latter embodiment, for example, centrifugation of the finished water sample out of the collection device can entail passage of the finished water through a chelation or sequestration matrix in route to a collection chamber which may or may not itself be provided as a component of the immunoassay device.

Additionally, pH levels in the testing solution can also interfere with molecular recognition due to its effect on the protonation state of acidic and basic groups on the surface of either the molecular recognition element and/or the targeted agent. Such chemical moieties that can be affected by pH include for example, histidine, carboxylic acid, amines, as well as others known in the art. This interference can be avoided, for example, by adding a buffering agent. Buffering agents are compounds whose solutions act to resist changes in pH from the addition of base or acid. The term “pH buffering agent” as used herein refers to any organic or inorganic compound or combination of compounds that will maintain the pH of a finished water sample or solution to within about 0.5 pH units of a selected pH value. A typical buffer consists of a weak acid and its conjugate base, and is chosen to operate in a particular pH range, or for other properties important to the buffered system. For example, phosphate buffers are commonly used to buffer solutions of phosphatase enzymes because they inhibit the catalytic properties of the enzymes. A pH buffering agent may be selected from, but is not limited to, Tris(hydroxymethyl) aminomethane (tromethaprim; TRIZMA base), or salts thereof, sodium and/or potassium phosphate, 2-(N-Morpholino)ethanesulfonic acid, 3-(N-Morpholino)propanesulfonic acid, N-2-Hydroxyethylpiperazine-N′-2-ethanesulfonic acid, Tris(hydroxymethyl)aminomethane, as well as phosphates or any other buffering agent that is physiologically acceptable in finished water. In one embodiment, the pH buffering agent is Tris(hydroxymethyl)aminomethane (TRIZMA Base), has a concentration in the antimicrobial solution of between about 10 mM and about 100 mM, and maintains the pH in the range of about 6.0 to about 9.0. While one of ordinary skill in the art will recognize that any physiologically acceptable concentration and pH value is within the scope of the present invention, in another embodiment the buffering agent is 50 mM Tris and maintains the pH value at about 7.0 to about 8.0.

A reducing agent can also be used. In one embodiment, the reducing agent is selected from the group consisting of dithiothreitol (DTT), thioglycerol, and mercaptoethanol. In one embodiment, the concentration of reducing agent is from about 1 mM to about 200 mM. In one embodiment, the buffering agent is sodium phosphate or sodium borate, at pH 6.5, is from about 15 mM to about 100 mM. In another embodiment, the chelating agent is ethylenediaminetetraacetic acid (EDTA). Preferably, the concentration of EDTA is from about 1 mM to about 10 mM. In another embodiment, the detergent is sodium dodecyl sulfate (SDS) or polyoxyethylenesorbitan monolaurate. Preferably, the concentration of detergent is from about 0.01% to about 0.5%.

Carriers can also be added to the testing sample. The term “carrier” as used herein refers to any pharmaceutically acceptable solvent of chemicals, chelating agents and pH buffering agents that will allow the composition of the present invention to be added to the finished water. A carrier as used herein, therefore, refers to such solvent as, but is not limited to, water, saline, physiological saline, ointments, creams, oil-water emulsions or any other solvent or combination of solvents and compounds known to one of skill in the art that is pharmaceutically and physiologically acceptable in finished water.

Additionally diluents can be added. Where a diluent is provided, suitable diluents are chosen to be compatible with the analyte and with the target antibodies and/or proteins in the subject assay. Typically the diluents are chosen to avoid denaturation or other degradation of the proteins or antibodies and to provide a milieu compatible with and facilitating of antibody/target (epitope) binding. While any diluent typically used in immunoassays is suitable (See, e.g., Current Protocols in Immunology Wiley/Greene, NY; Harlow and Lane (1989); Antibodies: A Laboratory Manual, Cold Spring Harbor Press, NY; Stites et al. (eds.) Basic and Clinical Immunology (4th ed.) Lange Medical Publications, Los Altos, Calif., and references cited therein), a particularly preferred diluent comprises 0.1M NaHCO3, pH 8.0. A preservative can also be added (e.g., about 0.01% thimerosal). Particularly preferred diluents are buffers ranging from about pH 7 to about pH 9, more preferably from about pH 7.5 to about pH 8.5, and most preferably around pH 8. One of skill in the art will appreciate that the diluent (sample buffer) can additionally include a protein or other moiety unrelated to the analyte which participates in non-specific binding reactions with the various components of the assay (e.g., the substrate) and thereby blocks and prevents non-specific binding of the antibodies. A particularly preferred blocking agent is bovine serum albumin (BSA) or polyvinyl alcohol (PVA). In one embodiment, the finished water sample is diluted at a diluent:sample ratio ranging from about 1:1 up to about 1:20 (v/v), more preferably from about 1:1 up to about 1:15 (v/v) and most preferably from about 1:1 up to about 1:10 (v/v). In one particular preferred embodiment, the sample is diluted at a diluent:sample ratio of about 1:8 (v/v). In certain embodiments, the finished water sample may not be diluted at all prior to use.

In another embodiment, the blocking agent of non-specific binding is gelatin or bovine serum albumin. Generally, the blocking agent of non-specific binding is gelatin. Preferably, the concentration of gelatin is from 0.05% to about 1.0%. In another embodiment, the chaotropic agent is sodium thiocyanate or ammonium thiocyanate. In another embodiment, the antigen diluent or buffer comprises 25 mM sodium phosphate, pH 6.5, 5 mM EDTA, 10 mM DTT, 0.2% gelatin, 100 mM ammonium thiocyanate, 0.09% sodium azide and 0.1% SDS.

By way of example, before completing an assay of the sample of finished water, the enviromnental conditions of the sample of interest may be adjusted by combining with an antigen diluent or buffer comprising one or more of the following: a reducing agent, a buffering agent, a chelating agent, a blocking agent of non-specific binding, a chaotropic agent, an antibacterial agent, and a detergent.

Further, to overcome the molecular recognition problems described above, the molecular recognition elements can themselves be manipulated so that they can accurately recognize the modified targeted agent. Such manipulation can occur, for example, by determining what affect the natural qualities and/or additives in the water have on the targeted agent. The molecular recognition elements can than be constructed to identify the targeted agent in its altered state. For example, new antibodies or nucleic acid sequences that have been manipulated to recognize the altered portions of the targeted agent can be used. These altered molecular recognition elements are then used in molecular recognition events as described above.

The natural qualities and/or additives included in water that are added during treatment can also interfere with transduction of the recognition even to a recognizable signal. The interference can be caused by, for example, the pH level of the testing solution, metal ions binding to and inhibiting recognition sites on the molecular recognition elements, as well as anions competitively inhibiting a label on a molecular recognition element (i.e., phosphate inhibiting a phosphatase enzyme label). For example, in an enzyme-based transduction, the constituents in the water can affect the effectiveness of the enzyme in creating an enzyme product. Without a resultant enzyme product, no signal and verification of the presence of the targeted agent can occur. In such a case, the testing solution should be manipulated so that the enzyme, or other transduction mechanism, can operate to provide the desired signal. The solution can be manipulated using methods known in the art such as, for example, by adding a buffer to modify the pH an ionic strength, adding essential metal ions for enzyme activity and suppressing inhibitory ones, or adding a mild reducing agent (i.e., thiosulfate) to neutralize residual chlorine and other disinfectants.

As used herein, “Biological Agent” is defined as any microorganism, pathogen, or infectious substance, toxin, or any naturally occurring, bioengineered or synthesized component of any such micro-organism, pathogen or infectious substance, whatever its origin or method of production. Such biological agents include, for example, biological toxins, bacteria, viruses, rickettsiae, spores, fungi, and protozoa, as well as others known in the art.

“Biological toxins” are poisonous substances produced or derived from living plants, animals, or microorganisms, but also can be produced or altered by chemical means. A toxin, however, generally develops naturally in a host organism (i.e., saxitoxin is produced by marine algae), but genetically altered and/or synthetically manufactured toxins have been produced in a laboratory environment. Compared with microorganisms, toxins have a relatively simple biochemical composition and are not able to reproduce themselves. In many aspects, they are comparable to chemical agents. Such biological toxins are, for example, botulinum and tetanus toxins, staphylococcal enterotoxin B, tricothecene mycotoxins, ricin, saxitoxin, Shiga and Shiga-like toxins, dendrotoxins, erabutoxin b, as well as other known toxins.

Bacteria are small, single-celled organisms that can generally be grown on solid or in liquid culture media. Most bacteria do not cause illness in human, but those that do generally cause illness by either invading tissue or producing poisons or toxins. Bacteria that can be harmful to humans are, for example, Brucella sp., Escherichia coli (O157:H7), Francisella tularensis, Vibrio cholerae, Corynebacterium diphtheriae, Burkholderia mallei, Burkholderia pseudomallei, Yersinia pestis, Salmonella typhosa, Bacillus anthrascis, Aerobacter aerogenes, Aeromonas hydrophila, Bacillus cereus, Bacillus subtilis, Bordetella pertussis, Borrelia burgdorferi, Campylobacter fetus, C. jejuni, Corynebacterium diphtheriae, C. bovis, Cytophagia, Desulfovibrio desulfurica, Edwardsiella, enteropathogenic E. coli, Enterotoxin-producing E coli, Flavobacterium spp., Flexibacter, Helicobacter pylori, Klebsiella pneumoniae, Legionella pneumophiia, Leptospira interrogans, Mycobacterium tuberculosis, M. bovis, N. meningitidis, Proteus mirabilis, P. vulgaris, Pseudomonas aeruginosa, Rhodococcus equi, Salmonella choleraesuis, S. enteridis, S. typhimurium, S. typhosa, Shigella sonnet, S. dysenterae, Staphylococcus aureus, Staph. epidermidis, Streptococcus anginosus, S. mutans, Vibrio cholerae, Yersinia pestis, Y. pseudotuberculosis, Actinomycetes spp., Streptomyces reubrireticuli, Streptoverticillium reticulum, and Thermoactinomyces vulgarisas well as others known in the art.

Under special circumstances, some types of bacteria form endospores that are more resistant to cold, heat, drying, chemicals, and radiation than the bacterium itself. Examples of such spores that can be harmful to humans as a source of the bacterium are, for example, Bacillus anthracis, Clostridium botulinum, as well as others known in the art.

Viruses are the simplest type of microorganism and consist of a nucleocapsid protein coat containing genetic material, i.e., DNA or RNA. Because viruses lack a system for their own metabolism, they require living hosts for replication. Most viruses do not respond to antibiotics. Viruses that can be harmful to humans are, for example, the Marburg virus, Junin virus, Rift Valley Fever virus, variola virus, Venezuelan equine encephalitis virus, yellow fever virus, Dengue viruses (DEN-1, DEN-2, DEN-3 and DEN-4), Ebola virus, Congo-Crimean hemorrhagic fever virus, Lassa virus, Machupo virus, Nipah virus, hantavirus, as well as other viruses known in the art.

Rickettsiae are obligate intracellular bacteria that are intermediate in size between most bacteria and viruses and possess certain characteristics common to both bacteria and viruses. Like bacteria, they have metabolic enzymes and cell membranes, use oxygen, and are susceptible to broad-spectrum antibiotics, but like viruses, they grow only in living cells. Although most rickettsiae can be spread only through the bite of infected insects and are not spread through human contact, Coxiella bumetii can infect through inhalation. Examples of rickettsiae that can be harmful to humans are, for example, Rickettsia prowazkeii, Coxiella bumetii, Rickettsia rickettsii, as well as others known in the art.

Fungi are single-celled or multicellular organisms that can either be opportunistic pathogens that cause infections in immunocompromised persons (i.e., cancer patients, transplant recipients, and persons with AIDS) or pathogens that cause infections in healthy persons. Examples of types of fungi that can be harmful to humans are, for example, Blastomyces dermatitidis, Aspergillus, Candida albicans, Coccidioides immitis, Histoplasma capsulatum, Cryptococcus neoformans, Mucorales, Paracoccidioides brasiliensis.

Protozoa are unicellular eukaryotic organisms that feed by ingesting particulate or macromolecular materials, often by phagocytosis. Most protozoa are motile by means of flagella, cilia or amoeboid motion. Examples of protozoan that can be harmful to humans are, for example, Cryptosporidium parvum, Cyclospora cayatanensis, Giardia lamblia, Entamoeba histolytica, Toxoplasma, Microsporidia, Trypanosoma brucei gambiense Trypanosoma brucei rhodesiense, Plasmodium vivax, Plasmodium malariae, Plasmodium ovale, Plasmodium falciparum, as well as others known in the art.

A prion is a protein particle that is capable of causing an infection or disease. Like viruses, prions are not capable of reproduction by themselves, but unlike viruses, prions do not contain genetic material (DNA or RNA). Further, prions have the uncanny ability to change their shape and cause a chain reaction that makes other proteins of the same type change their shape as well. Prions are known to cause a group of devastating neurological diseases known as transmissible spongiform encephalopathies (TSEs), such as, for example, Creutzfeldt-Jakob disease in humans, scrapie in sheep, or bovine spongiform encephalitis in domestic cattle, as well as others known in the art.

There are various types of assays, with the principal ones being competitive and non-competitive. Competitive assays may be heterogeneous or homogeneous, but non-competitive assays are homogeneous, and are frequently referred to as sandwich assays. In competitive assays, first molecular recognition elements “MR” (unlabeled) and second molecular recognition elements “MR*” (labeled) that recognize the same targeted biological agent are added to a testing sample for competitive equilibrium with the targeted sequence. Depending on the analyte and the configuration of the reaction vessel used, equilibrium can take several minutes to hours. Results, however, can be obtained before equilibrium is reached. The unbound MR and MR* are then rinsed from the tubes and a substrate “S” is added. The added S in combination with MR* causes a product “P” that can be detected and analyzed. At a fixed time, the sample is analyzed for the product P, whose concentration is proportional to the MR* in the reaction chamber if the product reaction is carried out under substrate saturation conditions. Alternatively, S can act to alter MR* to provide a signal that can be detected and analyzed. Because of the competitive binding, a typical standard plot of the current versus the concentration of MR has an inverse, linear relationship. Each label on a MR* can generate a large number of P molecules, which leads to an extremely low level of detection (i.e., high sensitivity) for the targeted agent.

In a sandwich assay, multiple molecular recognition events occur. A first molecular recognition event using a first molecular recognition element targets and captures a specific agent. The captured agent then undergoes a second molecular recognition event with a second molecular recognition element that can cause the emission of a signal such as, for example, a label. The label can either manipulate an added substrate to provide the desired signal, or can itself, be manipulated to provide a signal.

In general, a molecular recognition event occurs when a molecular recognition element identifies and interacts with a unique component of a targeted biological agent. Molecular recognition elements can be, for example, antibodies, aptamers, enzymes, nucleic acids, natural or engineered receptors, molecularly imprinted polymers, specific ligands to which the target might bind, as well as others known in the art. Recognition, however, has been accomplished principally by targeting sites on the surface of a biological agent (i.e., epitopes) that are recognized by antibodies (immunoassay), or alternatively, gene fragments using nucleic acid probes (nucleic acid-based assay). As used herein, nucleic acid-based (NA-based) assay refers to an assay that uses nucleic acid sequences unique to the targeted agent as molecular recognition elements that recognize and identify the targeted agent. The choice between antibody and NA-based technologies for a particular application is not necessarily clear-cut, however, some targets, such as, for example, toxins and prions, contain no nucleic acid. In such a case, antibody recognition can become the default recognition mechanism. Further, antibody-based systems are generally faster in detection, but tend to be less selective, and NA-based systems tend to be less robust, which can be an important consideration for field and remote use where environmental controls are less predictable. Additionally, the target in a NA-based system can be somewhat protected from solvent surrounding the targeted biological agent, which can introduce a slowing, preparation step.

In an immunoassay, the phrase “specifically binds to an analyte” or “specifically immunoreactive with,” when referring to an antibody refers to a binding reaction which is determinative of the presence of the analyte in the presence of a heterogeneous population of molecules such as proteins and other biologics (i.e., such as may be found in finished water). Thus, under designated immunoassay conditions, the specified antibodies bind to a particular analyte and do not bind in a significant amount to other analytes present in the sample. A variety of immunoassay formats may be used to select antibodies specifically immunoreactive with a particular analyte. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity.

Antibodies are generally large glycoproteins (MW ˜160,000) synthesized by an animal's immune system to identify external species that have invaded the animal and to label them for elimination. Animals have upwards of 10⁷ different antibodies, each capable of binding with a different target species. Thus, antibodies provide a large pool of highly selective biological reagents for a wide variety of species including chemicals such as toxins and infectious agents such as spores, bacteria, and viruses. The exceptional specificity an antibody has for its target antigen and the magnitude of the antibody/antigen binding constant (up to 10¹¹) have made immunoassay using antibodies a widely accepted diagnostic technique in the medical and/or clinical area. Because antibodies exist for many biological agents that can be used as bioterrorism agents, they provide a basis for a detection system to monitor for the presence of such agents in a sample.

Appropriate antibodies for a targeted biological agent can be made, for example, by injecting an animal with the targeted antigen, isolating, and copying the resultant antibodies. Although the animal route for producing antibodies is traditional and widespread, it is more difficult to provide antibodies from a highly infectious or toxic agent because the animal may die. Further, such dangerous agents are subject to strict handling restrictions. Appropriate antibodies may further be produced by constructing recombinant Fab (antibody binding fragments) into a phage display. This is essentially the expression of a combinatorial library of Fab peptides on the surface of a population of a phage that can then be selected based on their desired selectivity. Since these are recombinant, the vagaries of mono- or polyclonal antibody production in vivo are avoided. Alternatively, antibodies for a particular biological agent can be purchased, for example, from various known commercial vendors.

NA-based molecular recognition can occur via aptamers. Aptamers are single stranded DNA or RNA polynucleotides that bind molecular targets with high affinity and specificity that rivals the binding affinity and selectivity of antibodies. They are prepared by the Systematic Evolution of Ligands by Exponential enrichment (SELEX) process, which is a relatively new method for generating high affinity receptors that are composed of nucleic acids instead of proteins. SELEX is typically performed by synthesizing a random oligonucleotide library of greater than 20 bases in length, which is flanked by known primer sequences. Synthesis of the random region is achieved by mixing eqimolar amounts of all four nucleotides at each locus in the sequence. Thus, the diversity of the random sequence is maximally 4″, where n is the length of the sequence, minus the frequency of palindromes and symmetric sequences. The greater degree of diversity conferred by SELEX affords greater opportunity to select for oligonucleotides that form 3-dimensional binding pockets. Selection of high affinity oligonucleotides is achieved by exposing a random SELEX library to an immobilized target analyte. Sequences, which bind readily without washing away, are retained and amplified by the PCR for subsequent rounds of SELEX consisting of alternating affinity selection and PCR amplification of bound nucleic acid sequences. Four to five rounds of SELEX are typically sufficient to produce a high affinity set of aptamers. High affinity aptamers can be generated much more rapidly than antibodies. Additionally, typical aptamer screening libraries contain 10¹²-10¹⁵ separate sequences providing a high probability of finding selective, high affinity binders. For these reasons, aptamers have been used as molecular recognition elements in assays.

Furthermore, other molecular recognition elements known in the art can also be used to engage in molecular recognition and transduction events to identify indicate the presence of a targeted agent such as, for example, those technologies disclosed by Iqbal S.; Mayo, M.; Bronk, B.; Batt, C.; Chambers, J.; “A Review of Molecular Recognition Technologies for Detection of Biological Threat Agents”, Biosensors & Bioelectronics 15 (2000) 549-578, which is herein incorporated by reference in its entirety.

Once a molecular recognition event occurs and the appropriate targeted biological agents have been identified and/or captured, the recognition should be converted into a quantifiable signal. Transduction of molecular recognition into a quantifiable signal has been accomplished in various ways that can be either separate from, or combined with recognition of the targeted biological agent. The focus in a transduction event is not so much the selectivity that can be provided by the recognition element, but instead sensitivity combined with speed. Various techniques can be used for transduction including, for example, electro-chemiluminescence, luminescence, fluorescence, surface plasmon resonance and variants, flow cytometry, electrochemistry, and polymerase chain reaction (PCR), with emerging efforts in other optical methods, microcapillary electrophoresis and array technologies. The method of transduction often includes a detectable label. The label may include, but is not limited to, a chromophore, an antibody, an antigen, an enzyme, an enzyme reactive compound whose cleavage product is detectable, rhodamine or rhodamine derivative, biotin, streptavidin, a fluorescent compound, a chemiluminscent compound, derivatives and/or combinations of these markers. Providing a signal with any label is carried out under conditions for obtaining optimal detection of the molecular recognition element. Assays, in particular immunoassays, that utilize particulate moieties as detectable labels are well known to those of skill in the art. Such assays include, but are not limited to fluid or gel precipitin reactions, agglutination assays, immunodiffusion (single or double), immunoelectrophoresis, immunosorbent assays, various solid phase assays, immunochromatograpy (e.g., lateral flow immunochromatography) and the like. Method of performing such assays are well known to those of skill in the art (see, e.g., U.S. Pat. Nos. 4,366,241; 4,376,110; 4,517,288; 4,837,168; 5,405,784; 5,534,441; 5,500,187; 5,489,537; 5,413,913; 5,209,904; 5,188,968; 4,921,787; and 5,120,643; British Patent GB 2204398A; European patent EP 0323605 B1; Methods in Cell Biology Volume 37: Antibodies in Cell Biology, Asai, ed. Academic Press, Inc. New York (1993); and Basic and Clinical Immunology 7th Edition, Stites & Terr, eds. (1991)).

The methods of this invention are practicable with essentially any assay that uses a particulate moiety as a detectable label. The term particulate moiety is used to refer to any element or compound that is insoluble in the particular buffer system of the immunoassay in which it is utilized or which precipitates out of solution to form a detectable moiety. Typically particulate labels are detected (i.e., recognized as producing a “signal”) when they accrete, agglutinate, or precipitate out of solution to form a detectable mass (distinguishable from the non-accreted, agglutinated or solubilized form of the “particle”), most preferably in a discrete region of the assay medium. Microparticles or “microparticulate labels” are particles or labels ranging in size from about 0.1 nm (average diameter) to about 1000 nm, preferably from about 1 nm to about 1000 nm, more preferably from about 10 nm to about 100 nm, and most preferably from about 15 nm to about 25 nm. Preferred particulate labels include, but are not limited to, particles such as charcoal, kolinite, bentonite, red blood cells (RBCs), colloidal gold, clear or colored glass or plastic (e.g. polystyrene, polypropylene, latex, etc.) beads or microspheres.

Many transduction techniques involve amplification, by either amplifying the signal directly, such as, for example using an enzyme. An enzyme can be used to convert a non-active substrate into an active signal. Further, the use of enzyme amplification can make an assay extremely sensitive because each enzyme molecule can catalyze the production of thousands of product molecules. It is generally the product molecules that are being detected, and thus, large amplification of the output signal can be provided, which enables extraordinarily low levels of detection to be achieved for the targeted agent. For the above reasons, enzymes are commonly used as catalytic labels in transduction of a signal, but in principle any catalytic material can be used, such as an inorganic coordination compound. Alternatively, the target can be amplified, for example, using the polymerase chain reaction (PCR) for nucleic acid, which reduces the sensitivity demanded of the assay by increasing the effective concentration of the target.

In the assay techniques disclosed herein, a molecular recognition element functions to identify a unique component of a targeted biological agent and capture it. The molecular recognition element can be introduced to a sample suspected of having a targeted biological agent (testing sample) using any method known in the art. For example, the molecular recognition elements can be fixed to a solid phase that is non-moveable such as, for example, microwells, capillaries, cuvettes, beads, fibers, as well as others known in the art. In such a case, a testing sample can be introduced to a solid phase that has attached recognition elements. The target biological agent, if present, will be captured and held by the molecular recognition elements fixed on the non-moveable solid phase. Transduction of the captured agent into a signal can be completed while the molecular recognition elements are still fixed to the non-mobile solid phase. Such transduction will be discussed below.

Alternatively, the molecular recognition element(s) can be attached to a mobile solid phase, such as, for example, macro-, micro-, or nanobeads, dipstick, or other moveable solid phase known in the art on which an immunoassay can be performed. For example, at least one molecular recognition element attached to a moveable solid phase can be introduced into a testing sample. Alternatively, a testing sample can be introduced into a solution having at least one mobile solid phase with an attached molecular recognition element. If present, the targeted biological agent will be captured and held by the molecular recognition elements that are attached to the mobile solid phase. Once the targeted biological agents are captured, the final aspect of the immunoassay, transduction can occur.

Using a small, mobile solid phase such as microbeads is advantageous because their size allows them to be dispersed throughout a small testing sample to provide a large surface area to sample volume ratio that enhances the capture of the targeted biological agent by minimizing diffusional distances. Further, the microbeads can be used in small volumes, which reduces the dilution of the signal-providing product in the transduction and detection steps, and therefore, maximizes sensitivity.

The mobile solid phase component may further be magnetic, such as, for example, magnetic nano- or microbeads, which allow the mobile solid phase to be held and/or manipulated by magnets during an assay. In particular, magnetic nano- or microbeads permit the use of a microfluidic assay system where all of the steps can be automated to give near-continuous monitoring. The beads can be transported through channels by fluid flow, captured, and held at specific points by a magnet. An example of a magnetic microbead that can be used is, for example, the 2.8 micron diameter Streptavidin-coated M-280 Dynabeads from Dynal Biotech, Inc. in Great Neck, N.Y.

The molecular recognition elements, as described herein, can be fixed to the solid phase using any method known in the art, such as, physisorption by noncovalent interactions, covalent bonding, or using a molecular element attached to the solid phase to bond to the captured molecular recognition element, either directly or by means of any suitable configuration of biotin to avidin, streptavidin, neutravidin, or any others known in the art.

As previously mentioned, once a molecular recognition element is attached to a solid phase and a targeted biological agent has been identified and captured, either the captured biological agent, or its associated molecular recognition element can be manipulated so that a visible and/or quantifiable signal is present. For example, a signal can be provided by associating the previously captured biological agent with a secondary molecular recognition element that has an attached label, which can be manipulated to emit a signal. Once the secondary molecular recognition element captures the targeted agent, either the label can be manipulated to emit a quantifiable signal, or the label can act to manipulate an added constituent to cause the emission of signal. As previously mentioned herein, such manipulation can occur using, for example, an enzyme. An enzyme, for example, can be attached to a molecular recognition element as a label and react with an enzyme substrate to form an enzyme product that emits a signal. Alternatively, an enzyme substrate attached to a molecular recognition element can be manipulated by an enzyme to form an enzyme product that emits a signal. Alternatively, non-enzyme labels can be used to provide a signal, such as, for example, quantum dots, fluorophores, electrochemical labels, spin, chelated metal labels, liposome labels, radioactive labels, as well as others known in the art. Furthermore, the capture of a targeted agent can be detected without a label using methods such as surface plasmon resonance, scanning microscopies, microcantilevers, as well as other methods known in the art.

Many techniques can be used to detect a signal indicating the presence of a targeted agent. Of these, electrochemistry is an effective detection method when a recognition element is tagged with, for example, an electroactive metal label, an electroactive organic group, or an enzyme that generates an electroactive product. As used herein, electroactive product, electroactive metal label, or electroactive organic groups, refers to those products, metal labels, or organic groups that can be oxidized by the removal of electrons or reduced by the addition of electrons. Electrochemical detection involves an electrochemical cell consisting of at least two electrodes: a working electrode made of a conductive material, such as platinum, gold, or carbon; and a reference electrode, such as a silver wire coated with silver chloride or a saturated calomel electrode. A third electrode, an auxiliary or counter electrode, which is made from a conductive material (i.e., carbon or stainless steel), can also be used. For voltammetric detection, a potential is applied to the working electrode with respect to the reference electrode, and the resulting current is measured. Current arises from the direct transfer of electrons across the electrode/solution interface upon oxidation or reduction of an electroactive species. Electrochemical detection may further include the use of potentiometry, in which the potential between an indicating electrode and the reference is electrode is measured. Thus, the signal indicates the potential of the cell rather than the current. In such a case, the label or enzyme product need not be electroactive. Any method known in the art can be used to conduct an electrochemical detection. Some advantages of electrochemical detection include, for example, detection ability in complicated sample matrices, simple instrumentation, low detection limits, and disposable electrochemical cells.

For example, a secondary molecular recognition element can have an attached enzyme label. An enzyme substrate can be added to the sample containing the captured biological agent and enzyme label. The enzyme that is either added to the testing solution or attached to a secondary molecular recognition element will catalytically convert the substrate to an electroactive product. By way of further example, an enzyme label of, for example, beta-galactosidase can be attached to a secondary molecular recognition element that has captured a targeted agent. An enzyme substrate of, for example, p-aminophenylgalactosidase (PAPG) can then be added to the sample converting the enzyme substrate to p-aminophenol (PAP), which can be electrochemically detected by oxidation. Other enzyme label systems that are known in the art to produce electroactive products can also be used, such as, for example, the use of alkaline phosphatase (ALP) as an enzyme label that converts p-aminophenylphosphate (PAPP) to PAP, which is electrochemically detectable. Examples of some enzyme systems that have been used for electrochemical detection are shown in Table 1. Alternatively, non-enzymatic electrochemical labels can be used such as, for example, metal labels, ferrocenyl labels, as well as others known in the art.

TABLE 1
Common Enzyme-Substrate-Product Systems in Electrochemical Assays

| Enzyme label | Substrate | Product |
|---|---|---|
| Alkaline phosphatase | 4-aminophenyl phosphate | 4- |
| (ALP) | (PAPP) | aminophenol |
| | | (PAP) |
| ALP | 1-naphthyl phosphate | 1-naphthol |
| ALP | glucose-6-phosphate | Glucose |
| ALP | 4-hydroxynaphthyl-1- | dihydroxy |
| | phosphate (HNP) | naphthalene |
| ALP | 3-indoxyl phosphate | indigo blue |
| ALP | phenyl phosphate | Phenol |
| ALP | 5-bromo-4-chloro-3-indolyl | H₂O₂ |
| | phosphate ester | |
| ALP | 6-(N-ferrocenoylamino)2,4- | 6-(N-fer- |
| | dimethylphenyl phosphate | rocenoyl- |
| | | amino)-2,4- |
| | | dimethyl- |
| | | phenol |
| ALP + bi-enzymatic | phenyl phosphate | phenol |
| biosensor (tyrosinase & | | |
| glucose dehydrogenase) | | |
| ALP + bi-enzymatic system | NADP+ | NAD+ |
| (NADH oxidase & alcohol | | |
| dehydrogenase (ADH)) | | |
| Horseradish peroxidase | 3,3′,5,5′- | TMB (ox) |
| (HRP) | tetramethylbenzidine (TMB) | |
| HRP | hyrdroquinone | benzoqinone |
| HRP | redox Os⁺²-based polymer | Os⁺³ |
| Glucose-6-phosphate | NAD⁺ + glucose-6-phosphate | NADH |
| dehydrogenase | | |
| Galactosidase | 4-aminophenyl-beta-D- | 4-amino- |
| | galactopyranoside (PAPG) | phenol |
| | | (PAP) |

Fluorescence detection is also a commonly used technique to determine the presence of a targeted agent. Fluorescence detection is relatively easy when the fluorophore has a strong luminescence, i.e., when the fluorescence quantum yield is close to unity. In cases where the quantum yield is relatively low, the experimental conditions of fluorescence excitation wavelength, the fluorescence yield, solid angle of the detection optics, and efficiency of the detector all play important roles in determining the overall efficiency of the measurement. In general, the fluorescence methodology can be conducted, for example, using an enzyme label similar to those described above for electrochemical detection. Fluorescence detection methods include, but are not limited to, direct detection of enzyme label emitted fluorescence, detection of fluorescence polarization, detection of fluorescence by resonance energy transfer, detection by quenching of fluorescence, as well as others known in the art. For example, after the initial capture of a targeted agent, a secondary molecular recognition element with an attached enzyme label can recognize and capture a previously captured agent. An enzyme substrate can be introduced into the sample of captured biological agents. The enzyme label can then alter the substrate into an enzyme product that is detectable through fluorescence.

In such a case, various enzymes, such as, for example, ALP and beta-galactosidase can be a label on a molecular recognition element. For these two enzymes, there are multiple fluorescent substrates that can be used to provide adequate fluorescence for detection. For example, fluorescein diphosphate (FDP) reacts with ALP and cleaves both phosphate moieties of the non-fluorescent FDP to produce the highly fluorescent fluorescein dye, which is easily excitable in the visible region at 490 nm with fluorescence emission maximum at 514 nm. The fluorescence quantum yield of fluorescein is known to be pH dependent having a high yield at high pH levels makes FDP a desirable labeled alkaline phosphatase substrate. There are, however, alternative fluorescently labeled alkaline phosphatase substrates that are effective including, for example, 7-hydroxy-9H-(1,3-dichloro-9,9-dimethylacridin-2-one)-phosphate (DDAO-phosphate), 4-methylumbelliferylphosphate (MUP), 6,8-difluoro-4-methylumbelliferylphosphate (DiFMUP). Alternatively, beta-galactosidase can, for example, be used as an enzyme label that reacts with various enzyme substrates, including, for example, fluorescein di-beta-D-pyranoside (FDG), 4-methylumbelliferyl-beta-D-pyranoside (MU-gal), Resorufin beta-D-galactopyranoside (Resorufin-gal), DDAO beta-D-galactopyranoside (DDAO-gal), as well as other enzyme substrates known in the art.

Examples of various enzymes and the resulting fluorophore products and characteristics are listed below in table 2. Although the below enzymes, enzyme substrates, and enzyme products are listed, others known or developed may be used as well.

TABLE 2
Fluorophore Characteristics

| | | | Excitation | Emission | Fluorescence |
|---|---|---|---|---|---|
| | | | Wavelength | Wavelength | Quantum |
| Enzyme | Enzyme | Fluorescent | Maximum | Maximum | Yield (Aq. |
| Label | Substrate | Product | (nm) | (nm) | Sol., pH 9) |
| Alkaline | FDP | Fluorescein | 490 | 514 | High |
| Phosphatase | | | | | |
| Alkaline | MUP | MU | 360 | 449 | Medium |
| Phosphatase | | | | | |
| Alkaline | DiFMUP | DiFMU | 358 | 452 | Medium |
| Phosphatase | | | | | |
| Alkaline | DDAO- | DDAO | 646 | 659 | Medium |
| Phosphatase | phosphate | | | | |
| Beta- | FDG | Fluorescein | 490 | 514 | High |
| galactosidase | | | | | |
| Beta- | Mu-gal | MU | 360 | 449 | Medium |
| galactosidase | | | | | |
| Beta- | Resorufin-gal | Resorufin | 571 | 585 | Medium |
| galactosidase | | | | | |
| Beta- | DDAO-gal | DDAO | 646 | 659 | Medium |
| galactosidase | | | | | |

Because of the highly toxic and/or infectious nature of the various biological agents that can be detected by the methodology described herein, and especially bioterrorism agents, the limit of detection (LOD) is an important consideration, and ideally is as low as only one organism for some agents. LOD, however, can depend on assay conditions, as well as how the assay is completed. Further, LOD of an immunoassay is often determined by non-specific adsorption (NSA). NSA is broadly defined as the unwanted presence of a conjugate (i.e., the attachment of a first molecular recognition element and second molecular recognition element to one another) after the last rinsing step when the substrate is added to the conjugate/targeted agent/solid phase complex. These unwanted conjugates can, for example, attach themselves to an assay device, or to the solid phase used to attach molecular recognition elements.

The above-described phenomenon can have a large effect on LOD by contributing to a background signal that can be substantial if not controlled. The interaction is generally hydrophobic and may have an electrostatic component depending on the components involved. NSA is therefore commonly “blocked” by substances that compete more effectively for the adsorption sites than does the conjugate. Such blockers include gelatin, BSA, casein, ion pairing reagents, detergents, and combinations thereof. NSA is exacerbated by the assay requirement that the conjugate concentration be high to drive the association of a conjugate and targeted agent as close to completion as possible, thereby lowering the LOD. NSA also generally increases, and becomes increasingly less reversible, with time. Therefore, the exposure of the captured targeted agent on the solid phase to conjugate should be brief and at the highest concentration compatible with the desired LOD. Another important factor in determining LOD is the association constant between the first molecular recognition element and targeted agent. Large association constants (i.e., tight binding) provide more effective capture of the targeted agent by the first molecular recognition element and more sensitivity for detecting the captured agent when using a secondary molecular recognition element with an attached label, both of which provide lower limits of detection.

All publications mentioned herein are incorporated herein by reference for the purpose of describing and disclosing the compositions and methodologies which are described in the publications which might be used in connection with the presently described invention. The publications discussed herein are provided solely for their disclosure prior to the filing date of the present application. Nothing herein is to be construed as an admission that the invention is not entitled to antedate such a disclosure by virtue of prior invention.

While the present invention has been illustrated by description of several embodiments, and while the embodiments have been described in considerable detail, it is not the intention of the applicant to restrict, or in any way limit the scope of the appended claims to such detail. Additional advantages and modifications may readily appear to those skilled in the art.

1. A method of detecting biological agents in finished water, comprising the steps of:
analyzing a sample of finished water suspected of having a targeted biological agent;
determining the effect said finished water has on the targeted agent;
selecting at least one first molecular recognition element that identifies
providing a first recognition element manipulated to target a biological agent in finished water;
flowing at least one sample suspected of having said biological agent over the first recognition element;
capturing said biological agent present in the sample with the first recognition element; and
emitting a signal indicating the presence of the targeted biological agent in said sample.

2. The method of claim 1, further including the step of associating at least one second molecular recognition element to the captured targeted agent,
wherein said second molecular recognition element is manipulated to target the captured biological agent in finished water.

3. The method of claim 2, wherein either the first recognition element or the second recognition element comprises a label that is capable of causing the emission of said signal indicating the presence of the targeted biological agent in the sample.

4. The method of claim 3, wherein said label converts an added substrate to provide a product that emits a quantifiable signal.

5. The method of claim 3, wherein the signal emission is capable of electrochemical detection.

6. The method of claim 3, wherein the signal emission is capable of fluorescence detection.

7. The method of claim 1, wherein said first molecular recognition elements are selected from the group consisting of antibodies, nucleic acid probes, molecularly imprinted polymers, natural receptors, and engineered receptors.

8. The method of claim 1, further including the step of treating the sample to circumvent interference with a molecular recognition event between said first or second molecular recognition element and the targeted agent.

9. The method of claim 8, wherein treatment of the sample includes adding an additive to the sample selected from the group consisting of a buffering agent, a chelating agent, a reducing agent, metal ions, and combinations thereof.

10. The method of claim 1, wherein said method detects biological agents selected from the group consisting of bacteria, fungi, protozoa, rickettsiae, spores, toxins, and viruses.

11. The method of claim 1, wherein said first molecular recognition element is associated with a solid phase.

12. The method of claim 3, wherein said solid phase is non-mobile selected from the group consisting of capillaries, microchannels, cuvettes, beads, fibers, and combinations thereof.

13. A method of increasing assay detection of a biological agent in a finished water sample, the method comprising the step of
(a) providing a finished water sample; and
(b) adjusting the environmental conditions of the finished water sample of interest by combining the finished water sample with an antigen diluent or buffer comprising one or more compounds selected from the group consisting of a reducing agent, a buffering agent, a chelating agent, a blocking agent of non-specific binding, a chaotropic agent, an antibacterial agent, and a detergent; wherein the antigen diluent or buffer is present in a concentration sufficient to produce positives in the assay.

14. The method of claim 13, wherein the method further comprises the step prior to step (a) of determining the chemical make-up of the finished water sample wherein after determining the finished water sample, providing the one or more adjusting compounds according to the environmental conditions determined.

15. The method of claim 13, wherein the method further comprises the step of selecting a molecular recognition element for use in the assay that is capable of binding the biological agent within the determined environmental conditions.

16. The method of claim 13, wherein the buffering agent is at a concentration from about 15 mM to about 100 mM.

17. The method of claim 13, wherein buffer contains a reducing agent selected from the group consisting of dithiothreitol (DTT), thioglycerol, and mercaptoethanol.

18. The method of claim 13, wherein the concentration of reducing agent is from about 1 mM to about 200 mM.

19. The method of claim 13, wherein the pH of the final solution is in the range of about 6.0 to about 9.0.

20. The method of claim 13, wherein the chelating agent is in a concentration of from about 1 mM to about 100 mM.

21. The method of claim 13, wherein the concentration of detergent is from about 0.01% to about 0.5%.

22. The method of claim 13, wherein the antigen diluent or buffer comprises 25 mM sodium phosphate, pH 6.5, 5 mM EDTA, 10 mM DTT, 0.2% gelatin, 100 mM ammonium thiocyanate, 0.09% sodium azide and 0.1% SDS.
